(19) United States
(12) Patent Application Publication
(10) Pub. No.: US 2011/0118570 A1
(43) Pub. Date: May 19, 2011

(54) OPTIC SENSOR DEVICE WITH SERS

(75) Inventor: Hans Joergen Pedersen, Harrislee (DE)

(73) Assignee: P & V Consulting GmbH & Co. KG, Ludwigsburg (DE)

(21) Appl. No.: 12/935,490

(22) PCT Filed: Mar. 31, 2009
(86) PCT No.: PCT/DK09/00078

§ 371 Date: Jan. 13, 2011

(30) Foreign Application Priority Data
Mar. 31, 2008 (DK) PA 2008 00467

(51) Int. Cl. A61B 5/1455

(52) U.S. Cl. 600/316

(57) ABSTRACT

The present invention relates to a system for determining a concentration of a substance of interest in a body fluid, more specifically to systems comprising sensor devices for optically detecting compounds such as glucose. The system of the present invention is, e.g., suitable for continuous determination of a concentration of a substance of interest which is present in a body fluid, e.g. a concentration of glucose in blood or interstitial fluid.

### CROSS REFERENCE TO RELATED APPLICATIONS

This application is entitled to the benefit of and incorporates by reference essential subject matter disclosed in International Patent Application No. PCT/DK2009/000078 filed on Mar. 31, 2009 and Danish Patent Application No. PA 2008 00467 filed on Mar. 31, 2008.

### TECHNICAL FIELD

The present invention relates to a system for determining a concentration of a substance of interest in a body fluid, more specifically to systems comprising sensor devices for optically detecting compounds such as glucose. The system of the present invention is, e.g., suitable for continuous determination of a concentration of a substance of interest which is present in a body fluid, e.g. a concentration of glucose in blood or interstitial fluid.

### BACKGROUND

Diabetic patients can improve their life quality and life expectancy by maintaining their blood glucose concentration close to the natural level of a healthy person. To achieve this natural concentration, diabetic patients must frequently measure their glucose concentration, and adjust their insulin dosing in accordance with the measured concentration. Usually, a blood sample is obtained for measurement of blood glucose concentration, and there are a number of different glucose test kits on the market based on measurement from a blood sample. The disadvantage of these test kits is the need to take a blood sample which must be collected from a suitable place in the body.

Self monitoring devices, based on capillary blood glucose, are practical but still require repeated and frequent skin punctures, which is inconvenient for the patient and requires certain hygienic precautions.

Biological sensors in the form of implantable devices are also known in the art and include electrochemical devices and optical devices based on the creation of an electrical or optical signal by the consumption of the compound detected by the analysis. An example is to be found in U.S. Pat. No. 6,011,984, which discloses methods utilising an amplification component. The sensitivity and the responsivity of such devices are influenced by the formation of a bio film, for example, by fibrous encapsulation of the device which reduces the transport rate of the compound to the sensor. Depending on the specific sensor, other mechanisms which cause deterioration of sensor performance of sensor devices, may also be present, for example, membrane de-lamination and degradation, enzyme degradation and electrode passivation.

Various proposals have been made for non-invasive measurement of glucose levels in the human body by spectroscopic methods but the effects of water in the body, the low concentration of glucose to be measured and the optical effects produced by skin all contribute to the difficulty of making satisfactory measurements.

Among the non-invasive optical methods for measuring blood glucose concentrations are WO 2007/072300 and WO 2006/003551.

WO 2007/072300 discloses a system and method for non-invasive measurement of glucose concentration in a live subject including a thermal emission spectroscopy (TES) device, an optical coherence tomography (OCT) device or near infrared diffuse reflectance (NIDR) device. The TES generates a signal indicative of the absorption of glucose, from which the blood glucose concentration is determined and the OCT device generates a signal indicative of the scattering coefficient of a portion of the live subject, from which the blood glucose concentration is determined.

WO 2006/003551 discloses a spectroscopic system for non-invasive spectral analysis of substances or biological structures that are located in a plurality of various volumes of interest. The spectroscopic system makes use of a multiplicity of various probe heads that are connected to a base station providing a spectroscopic light source and spectroscopic analysis means.

The systems disclosed in WO 2007/072300 and WO 2006/003551 both suffer from the drawback that non-invasive optical measurements of concentrations of substances, such as blood glucose, are less accurate than invasive measurements, and thereby the accuracy of the measurements is sacrificed in order to make the measurement easier to perform by the user.

U.S. Pat. No. 7,277,740 discloses a system for reagent-free determination of the concentration of an analyte in vivo. The system comprises a light transmitter for generating monochromatic primary light, a scattered light percutaneous sensor which includes an inbound light guide and a detection light guide, a wavelength-selective detection device that is connected to the detection light guide for detection of Raman-scattered components of the secondary light and an evaluation device for determining the concentration of the analyte from the Raman-scattered components of the secondary light.

### SUMMARY

It is an object of the invention to provide a detector, or sensor device, and a system which can be used for monitoring analyte concentration but enables the effects of the tissue in the analysis to be reduced.

The present invention provides a system for determining a concentration of a substance of interest in a fluid, possibly but not excluded to body fluid, the system comprising:

a sensor device (or detector) adapted to be positioned in direct contact with a body fluid to be analysed, said sensor device defining an analysis volume, at least partly delimited towards the body fluid by a semi-permeable membrane allowing substances of interest from the body fluid to enter the analysis volume,
a light source adapted to transfer primary light to the sensor device,
wherein the sensor device comprises at least one substantially flat area constituting a first area and at least one substantially flat area constituting a second area, at least one of the first and/or second area comprising an optically responsive element.

The optically responsive element in the following context means any device or element actively or passively reacting to light for example a photo detector or just a surface reflecting a part or all of the light hitting the surface.

The difference between the said distances allows a differential analysis to take place so that the effects of skin in the results can be reduced or avoided. The differential analysis may be a simple formation of a difference in signals or a complex computer correlation. The analysis may be performed within the device or externally.

The detector or sensor device may be divided into areas at different levels. By this means, the distance travelled by the light through the compound of interest in the body tissues, and thus the interaction of light with the compound, varies from area to area.

The spacing between the different levels may, for example, be between 0.5 and 5 millimetres, for example, between 0.5 and 3 millimetres, for example between 1 and 2.5 millimetres.

Preferably, groups of areas are provided, each group of areas forming a common level, the common levels having a predetermined spacing from each other to provide the said difference in distance over which light interacts with compounds and tissue.

Preferably, the first group of areas is formed at a base level of the device and the second group of areas is formed by projections from the said base level to a top level.

In one preferred embodiment of the present invention, at least one optically responsive element is a reflective surface being a structured metal surface, e.g. a micro structured metal surface. The structured metal surface is preferably of a kind which is suitable for adsorption of molecules of the substance of interest. In this case an enhanced Raman signal can be obtained by means of Surface Enhanced Raman Spectroscopy (SERS). Thereby it is possible to detect the presence of substances of interest even at very low concentrations.

The structured metal surface may advantageously be made from a precious metal, such as gold, silver, copper or platinum.

In order to direct light to and/or from the sensor device, the system in one embodiment further comprises first light guiding means arranged to guide primary light from the light source to the analysis volume, and/or second light guiding means arranged to guide secondary light from the sensor device.

In another embodiment, the sensor device may include means to provide a differential analysis of signals arising from the said difference in distances. Alternatively in yet another embodiment, a differential analysis may be performed on the data in an external apparatus to which the device transmits data.

In order to inhibit influences such as temperature dependence on the measurements, heating means and/or cooling means may be provided to act upon the body region surrounding the device.

The areas may be provided within wells to reduce the effects of stray light.

The areas may be covered by optical filter means to prevent light of wavelengths other than those of interest from reaching the areas.

The system may comprise focusing means such as a lens, to focus light to and/from the sensor device.

In a further embodiment, at least one substantially flat area constituting a third area or group of areas may be provided and form a common level between the base level and the top level.

At least a part of at least one of the first, second and/or third areas may be formed by a permeable membrane, where the membrane in one embodiment may be permeable to glucose.

In another aspect, the invention relates to one kind of method for optically detecting the content of a compound in the body of a living creature, the method comprising:
directing a light source on different areas of an sensor device containing detection means;
obtaining light-representing signals by means of said detection means;
transmitting said light-representing signals to an external device; and analysing the detected signals to obtain a value for the content of the compound to be detected.

Such methods allow non-invasive measurement after the initial implantation in which the influence of bio fouling on the value of the measured signal is decreased.

However, the methods are also suitable for invasive measurements.

The analysis of the detected signals may be a differential analysis.

The compound may be glucose.

Alternatively, the sample may be obtained separately and subsequently be positioned in direct contact with the sensor device. In the latter case the sample may be obtained by means of a separate apparatus and subsequently supplied to the system of the invention.

Alternatively the sample may be collected by means of a catheter or the like which is connected directly to the system of the invention, in which case the sampling equipment and the system of the invention form part of the same apparatus, the system of the invention itself being arranged non-invasively.

As written above, the sensor device preferably defines an analysis volume which is at least partly delimited towards the body fluid by a semi-permeable membrane. In the present context the term ‘semi-permeable’ should be interpreted to mean that some substances are allowed to pass the membrane while other substances are not allowed to pass. Preferably, the membrane allows substances of interest, such as glucose, to pass, while other elements or constituents of the body fluid are substantially not allowed to pass. Accordingly, the substance of interest passes through the semi-permeable membrane and into the analysis volume, while other constituents of the body fluid are kept out of the analysis volume, and thereby analysis of the substance of interest can take place in the analysis volume in order to determine the concentration of the substance of interest.

The sensor device is generally the part of the system which is arranged in direct contact with the body fluid to be analysed during operation of the system, and could be a part of a probe head, but it could alternatively be a part of the system which does not form a head or an end part. For instance, the analysis part could be a part arranged on the middle of an optical fibre. The analysis volume is defined by the analysis part, preferably forming a part of the analysis part.

The system further comprises first light guiding means and second light guiding means. The first light guiding means is arranged to guide primary light to the analysis volume, and the second light guiding means is arranged to guide secondary light away from the analysis volume. The first light guiding means and the second light guiding means may be completely separated, e.g. in the form of two separate light guides, each guiding light to/from the analysis volume. Alternatively, the first and second light guiding means may be at least partly combined, e.g. in the form of a Y-shaped structure in which only one light guide enters the analysis volume, said light guide being used to guide primary light into the analysis volume as well as to guide secondary light away from the analysis volume. In this case the light guide is split outside the analysis volume into a first light guide to guide primary light towards the combined part of the light guide and a second light guide to guide secondary light away from the combined part of the light guide.

The system of this embodiment is preferably operated in the following manner. The sensor device is arranged in contact with the body fluid, thereby allowing the substance of interest to enter the analysis volume via the semi-permeable membrane. Primary light is supplied to the analysis volume by means of the first light guiding means, thereby causing scattering, e.g. Raman scattering, on the molecules of the substance of interest present in the analysis volume. The secondary light produced in this manner is guided away from the analysis volume via the second light guiding means. Analysing the spectrum of the secondary light, e.g. the Raman spectrum, can then be used to determine the concentration of the substance of interest.

Thus, a system is provided which is adapted to provide precise determination of the concentration of a substance of interest. Furthermore, the system may easily be used in combination with a continuous sampling system, such as a catheter, and there is therefore no need to penetrate the skin each time it is necessary to perform a measurement.

The system may further comprise at least one laser, or another suitable light source being adapted to emit substantially monochromatic light, for emitting primary light, said laser(s) being connected to the first light guiding means. According to this embodiment the primary light is provided by means of at least one laser, and the laser(s) form(s) part of the system. The laser(s) may be permanently attached to an end of the first light guiding means which is arranged substantially opposite the analysis volume, or it/they may be connectable to the first light guiding means. Alternatively, a laser, or any other suitable primary light source, may be connected to the system without forming a part of the system. In this case the light source may be exchangeable and/or it may be selected by the user, e.g. to match a specific need, such as analysis of a specific substance of interest.

In the case that the system comprises at least one laser, at least one of the laser(s) may be a pulsed laser. The pulsed laser may advantageously be adapted to emit pulses having a duration which is shorter than 1 ps, such as shorter than 100 fs, such as in the femtosecond range. It is an advantage to use a pulsed laser which is adapted to emit pulses having a very short duration for the following reason. The shorter the duration of a laser pulse is, the broader a wavelength range is covered by the laser pulse. Thus, using a pulsed laser having a sufficiently short duration provides a wavelength profile which is sufficiently broad to at least substantially cover the entire Raman spectrum of the substance of interest. Thereby it is not necessary to scan the wavelengths or tune the input light source in order to obtain the Raman spectrum.

Alternatively or additionally, at least one of the laser(s) may be a continuous wave (cw) laser. According to this embodiment a cw laser may be used as a pump laser for enhancing population of Raman levels of molecules of a substance of interest. Thereby the signal used to determine the concentration of the substance of interest is enhanced, and the signal to noise ratio is improved. Thereby a more reliable determination of the concentration of the substance of interest is obtained. According to this embodiment the concentration of the substance of interest may be determined using coherent anti-Stokes Raman spectroscopy (CARS).

The sensor device may be adapted to be positioned invasively, such as subcutaneously or in a blood vessel, e.g. intravenously or intra-arterially, possible forming part of a probe head. It is an advantage that the probe head is adapted to be positioned invasively, since the sensor device is in this case arranged close to the sampling position. Thereby the response time of the system can be minimised.

In the case that the sensor device is adapted to be positioned subcutaneously, the body fluid may advantageously be interstitial fluid, and in the case that the probe head is adapted to be positioned in a blood vessel, the body fluid may advantageously be blood.

The system may further comprise detection means adapted to detect Raman scattered light, said detection means being connected to the second light guiding means. According to this embodiment the detection means used to detect, and possibly analyse, the Raman scattered light forms part of the system.

The optically responsive element(s) of the sensor device may further comprise at least one reflective surface arranged in an interior part of the analysis volume. According to this embodiment the primary light entering the analysis volume is reflected from the reflective surface. Thereby it travels a distance inside the analysis volume which is approximately twice as long as the distance travelled in the case where no reflective surface was present. Thereby the probability that the primary light is scattered from a molecule of the substance of interest is increased, and an enhanced signal can be obtained.

At least one of the reflective surface(s) may be provided with a structured metal surface, e.g. a micro structured metal surface. The structured metal surface is preferably of a kind which is suitable for adsorption of molecules of the substance of interest. In this case an enhanced Raman signal can be obtained by means of Surface Enhanced Raman Spectroscopy (SERS). Thereby it is possible to detect the presence of substances of interest even at very low concentrations.

The structured metal surface may advantageously be made from a precious metal, such as gold, silver, copper or platinum.

The metal microstructure may be formed in a number of various manners. As mentioned above, it may be a monolayer grown directly on an end part of a light guiding means. Alternatively, it may be in the form of nano-particles applied to an interior part of the analysis volume. As another alternative, the metal microstructure may be applied directly onto an end part of a light guiding means, e.g. by means of sputtering, chemical vapour deposition (CVD) or another suitable technique. As yet another alternative it may be a patterned metal layer applied to an end part of a light guiding means, e.g. using a masking technique or a photolithographic technique. Finally, it may be a semitransparent metal layer applied to an end part of a light guiding means using a suitable technique.

The first and/or the second light guiding means may comprise an optical fibre. Alternatively or additionally, the first and/or the second light guiding means may be or comprise any other suitable means to guide an appropriate kind of primary/secondary light to/from the analysis volume.

The substance of interest may be glucose. In this case the system of the invention may advantageously be used to measure the blood glucose level, e.g. in order to determine an amount of a drug, e.g. insulin, to be administered to a person, e.g. a person having insulin-dependent diabetes.

The body fluid is blood, interstitial fluid, or any other suitable body fluid containing the substance of interest.

The semi-permeable membrane may form part of the first light guiding means and/or the second light guiding means. This embodiment may advantageously be realised by providing a hollow fibre made from a semi-permeable material, the hollow fibre thereby constituting the semi-permeable membrane. An optical core may then be positioned in the interior of the hollow fibre in such a manner that a space is defined between the optical core and the semi-permeable membrane. This space may contain air or a suitable liquid, e.g. a saline solution. By selecting the material of the optical core in such a manner that the optical core has a higher index of refraction than the material contained in the space defined between the optical core and the semi-permeable membrane, the optical core, the hollow fibre and the material arranged between the optical core and the semi-permeable membrane in combination form an optical wave guide.

As an alternative to arranging the sensor device at an end part of the first and/or the second light guiding means, the sensor device may be arranged between the first light guiding means and the second light guiding means. According to this embodiment, primary light is guided to the sensor device via the first light guiding means, and secondary light is guided away from another, oppositely arranged, part of the sensor device via the second light guiding means.

The first light guiding means and the second light guiding means may form part of the same optical fibre. According to this embodiment the primary light and the secondary light may be guided by the same optical fibre. Alternatively, the analysis part may be arranged at a middle part of the optical fibre. In this case the primary light is guided to the analysis volume via a first part of the optical fibre, and the secondary light is guided away from the analysis volume via a second part of the optical fibre.

The system may further comprise at least one reflective surface arranged in an interior part or adjacent to the analysis volume. As described above, the primary light is thereby caused to pass through the analysis volume twice, thereby increasing the probability of the primary light being scattered on a molecule of the substance of interest.

The analysis volume may be formed by the sensor device. According to this embodiment the sensor device is arranged in direct contact with the body fluid to be analysed. In the case that the analysis part is to be positioned invasively, the sensor device should preferably be made from a biocompatible material, i.e. a material which is compatible with the kind of tissue in which it is intended to arrange the analysis part.

Devices constructed in accordance with the invention and methods in accordance with the invention will now be described, by way of example only, with reference to the accompanying drawings, in which:

### BRIEF DESCRIPTION OF THE DRAWINGS

FIG. 1 is a schematic representation showing an optical device, a sensor device and a receiving device;

FIG. 2 is a schematic representation of a first implantable device embodying the invention, the device containing optically responsive element(s) and being coated with a biocompatible material;

FIG. 3 is a schematic representation of a second implantable device embodying the invention, the device being step-like and having two detector levels;

FIG. 4 is a schematic representation of a third implantable device embodying the invention, the device containing a multiplicity of detection areas at two different levels;

FIG. 5 is a schematic representation of a fourth implantable device embodying the invention, the device having optically responsive element(s) placed in detector wells;

FIG. 6 is a schematic representation of a fifth implantable device embodying the invention, the device having detection areas covered by a membrane on the top;

FIG. 7 is a schematic representation of a sixth implantable device embodying the invention, the device having detection areas covered partly by a membrane and partly by a lid on the top;

FIG. 8 is a schematic representation of a seventh implantable device embodying the invention, the device including electric connections and an electronic circuit device;

FIG. 9 is a schematic representation of an eighth implantable device embodying the invention the device including a spacer covering some of the areas of the device;

FIG. 10 shows a section through FIG. 9 along the line X-X and shows the spacer.

FIG. 11 shows a system with a probe head enclosing the sensor device according to a first system embodiment.

FIG. 12 shows a system with a probe head enclosing the sensor device according to a second system embodiment.

FIG. 13 shows a system with a probe head enclosing the sensor device according to a third system embodiment.

FIG. 14 shows a system with a probe head enclosing the sensor device according to a fourth system embodiment.

FIG. 15 shows a system with a probe head enclosing the sensor device according to a fifth system embodiment.

FIG. 16 shows a system with a probe head enclosing the sensor device according to a sixth system embodiment.

FIG. 17 shows a system with a probe head enclosing the sensor device according to a seventh system embodiment.

FIG. 18 shows a system with a probe head enclosing the sensor device according to an eighth system embodiment.

FIG. 19 shows a system with a probe head enclosing the sensor device according to a ninth system embodiment.

FIG. 20 shows a system with an implanted sensor device according to a further system embodiment.

FIG. 21 shows a system with an implanted sensor device according to a further system embodiment.

### DETAILED DESCRIPTION

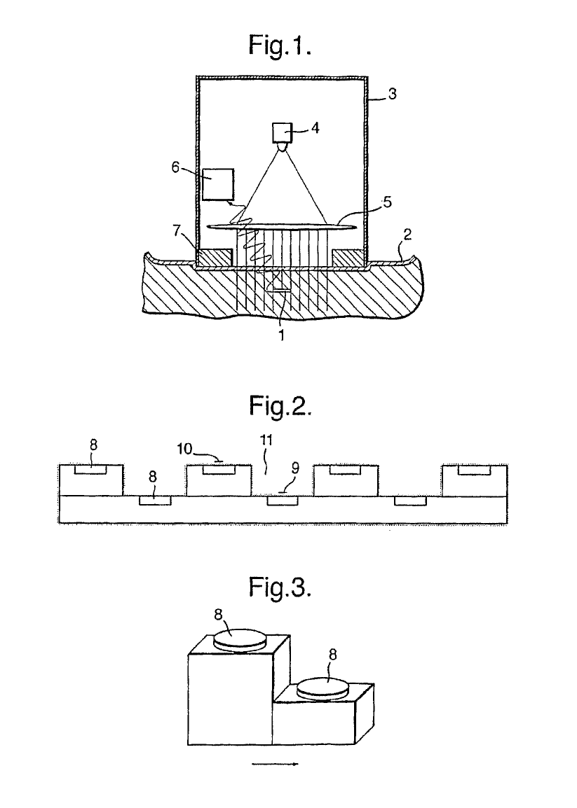

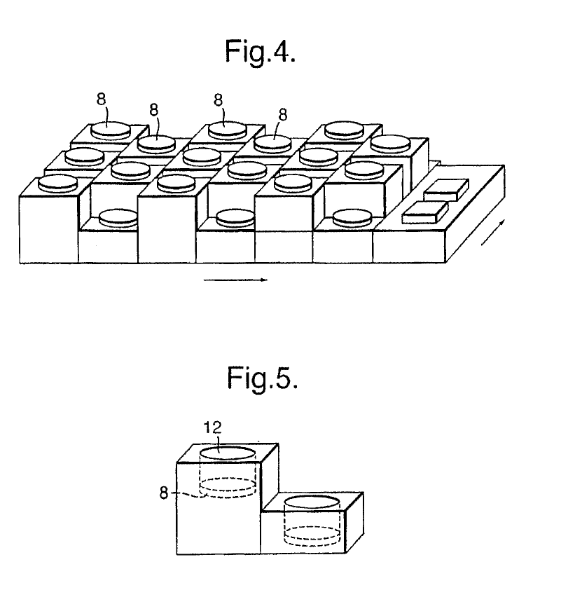

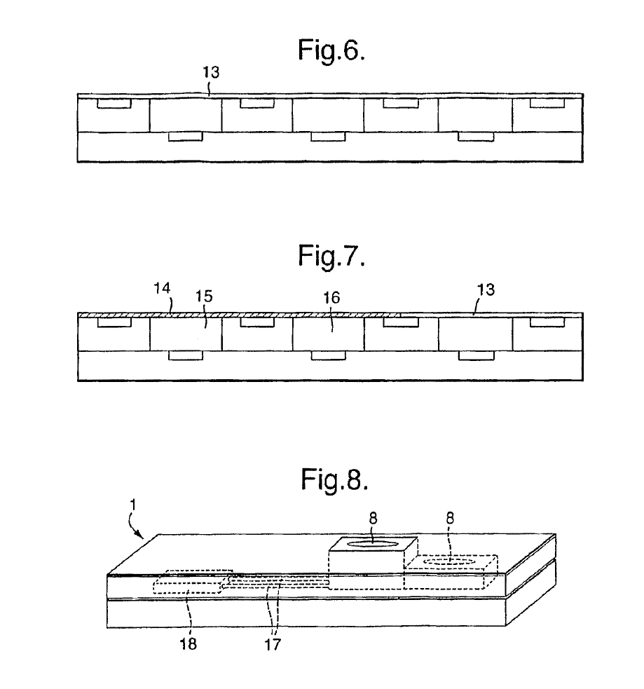

FIG. 1 illustrates a detector, or sensor device, as disclosed in EP 1 455 641, where light from a light source is incident on an implanted detector or sensor device (1), the light being detected by a detection device in the implanted detector or sensor device (1), and a signal being transmitted to a receiving device for analysis. The characteristics of the detected light depend on the interaction with the compounds encountered on the way from the light source to the sensor device (1).

The implanted sensor device (1) is divided into areas at different levels, so that the distance for the light through the compounds, —and thus the interaction with light, varies from area to area. A differential analysis is performed on signals produced by the detector.

The sensor device (1) is placed underneath the skin (2) so that the compound to be measured is contained between the skin and the sensor device. An optical device (3), containing a light source (30) and a lens system (5), is placed external to the skin above the sensor device, and a signal for the detected light is transferred from the sensor device (1) to a receiver (6).

The light intensity emitted from the light source is preferably approximately constant over the whole of the sensor device. It is thereby ensured that variations in the detected light are due only to absorption in the path from light source to detector and not due to variations in emitted light intensity.

Referring still to FIG. 1, the light source is, for example, a light source of a broad continuous spectrum, for example a thermal white light source, depending on the compound to be measured. In the case of measuring glucose concentration, the wavelength should be well represented in the near infrared spectrum, more specifically between 1000 and 2500 nm. The light source is in this case therefore, for example, an LED, one or more laser diodes or an LED array producing wavelengths in this range. Alternatively a monochromator can be used with a white light source to select light within a desired wavelength range and directed onto the sensor device.

Wavelength specific light detection can also be obtained by covering the detectors with a film, transparent for only a specific wavelength or wavelength range. In this way is it possible to detect within a range of wavelengths simply by having a light source with a range of wavelengths and a number of detectors with different films. The film covering each detector also prevents detection of background light, as this will not pass through the film. Alternatively the detection within a range of wavelengths can be carried out by having more than one light source, and successively directing light of different wavelengths on the sensor device.

The light absorption for some compounds is temperature dependent, meaning that the detected light on the sensor device varies with the temperature of the compounds and tissue. FIG. 1 shows a cooling/heating device (7), such as a Pelletier element, formed as a ring around the light emitting area. With this element it is possible to perform measurements at different temperatures, so as to facilitate and improve the analysis. In case of analysis at different temperatures, the actual temperature can be recorded by a thermo element or the like, placed in the device (3).

The present invention relates to such a sensor device (1), also seen in FIG. 2, where the device comprises optically responsive element(s) (8), and the present invention further relates to different systems wherein this sensor device (1) is used.

In the following context the optically responsive element (8) means any device or element actively or passively reacting to light, for example a photo detector, or just a surface reflecting part or all of the light hitting the surface.

In a preferred embodiment the present invention relates to the sensor device (1) comprising reflective surface(s) that may be provided with a structured metal surface, e.g. a micro structured metal surface. The structured metal surface is preferably of a kind which is suitable for adsorption of molecules of the substance of interest. In this case an enhanced Raman signal can be obtained by means of Surface Enhanced Raman Spectroscopy (SERS). Thereby it is possible to detect the presence of substances of interest even at very low concentrations.

Referring now to FIG. 2, the detector or sensor device (1) (in the following referred to as the sensor device (1)) shown here consists of a number of such optically responsive elements (8), optionally contained in a polymeric or elastomeric matrix with biocompatible surfaces. The shape of the sensor device (1) is made step-like to provide two levels of detection areas, base level (9) and extended level (10). In this way the detected light varies depending on the level in which the light is detected, and the variation is dependent on the interaction of light with compounds and components in the volume between the two levels, henceforth called the measurement volume, or analysis volume, (11).

The sensor device (1) can, for example, have a step-like shape in one direction, as indicated by an arrow in FIG. 3, or a step-like shape in two directions as indicated by two arrows in Fig, 4. Having more than one optically responsive element (8) at each level increases the sensitivity of the analysis, as the signals from each level can then be averaged.

Referring now to FIG. 5, each optically responsive element (8) is shown to be placed in a detection well (12) so that only parallel light is detected. This has the effect that only the emitted and directly transmitted light is detected and not light from another light source, such as background light.

Covering the sensor device (1) with a membrane (13) can reduce interference by other compounds and noise due to scattering components. The membrane is sufficiently transparent at the appropriate wavelengths employed for the measurement (if placed on top of the measurement volume), and is permeable to the compound to be measured, for example glucose, but prevents other molecules larger than the compound to be measured, from entering the measurement volume. The membrane can be placed above the measurement volumes, that is, between measurement volumes and the light source and detector, and/or to the sides of the measurement volumes. Placing the membrane to the side of the measurement volume enables a long optical path length and at the same time a relatively short response time of the device with respect to changes in the concentration in the surrounding tissue and liquid. This is because a larger membrane area is available for permeation into the measurement volume and because the required diffusion length of the compound in the measurement volume can be shorter than the optical path length. The measurement volume can be filled with liquid or with a solid matrix permeable to a compound to be analysed.

The detected signal of the device is calibrated to a known concentration of the compound to be analysed, either once and for all or preferably from time to time. Measuring the concentration in a sample, taken at the same time as the optical measurement, can be used to achieve this calibration. The device, however, can be made self-calibrating, if two measurement volumes contain a known concentration of compound.

In FIG. 7 a part of the device is covered with a diffusion proof lid (14) instead of with a membrane. This forms two measurement volumes, or analysis volumes, (15), (16) with known concentrations of the compound under analysis, preferably one volume with a concentration in the lower end and one volume with a concentration in the high end of the required measurement range.

The formation of a bio film on the sensor device will have less effect than is the case for electrochemical devices or other devices in which the compound to be measured is consumed in the measurement process. As long as the bio film is sufficiently transparent at the optical wavelengths employed, the bio film will have very little effect on the measurement. In the case where a membrane is used, as described above, the bio film may influence the response time with respect to changes in the concentration in the surrounding tissue and liquid, but it will still have little effect on the measurement itself.

The two levels of detection areas can be increased to three or more different levels. By increasing from two to three or more levels, the dynamic range of the sensor can be increased, as the analysis of the detected signal then discloses three or more levels corresponding to two or more interaction volume optical path lengths. Also more information is made available for data analysis to establish compound concentrations using, for example, chemometric, multivariant data analysis approaches. More levels also facilitate consistency and quality control of data.

Referring to FIG. 8, the sensor device, or detector, (1) of EP 1 455 641contains a number of optically responsive elements (8) being photo detectors, which are connected to an electronic circuit device (18) by wires (17). The electronic circuit device can be operated by power and data transmission without the use of connecting wires to the outside. Such power transmission can be implemented by the use of a so-called inductive link, which is basically a coreless transformer. Transmission of data from the electronic circuit device to the external receiver can take place, for example, by varying the load seen by the secondary of the transformer located in the sensor device (for example, the resistance change of a photo conductivity cell), or for example, by measuring the change of resonance frequency of a series resonant circuit (for example, the change of capacitance due to the photo current in a photo diode).

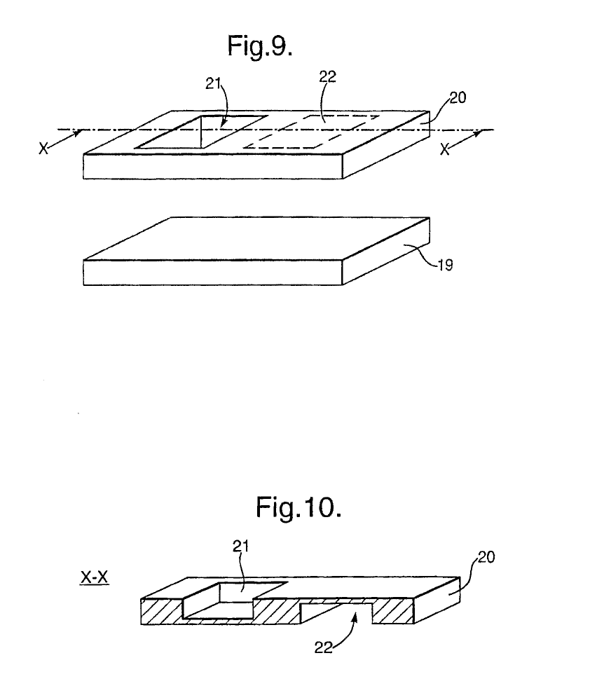

Turning now to FIG. 9, the sensor device is shown to be made as a laminated structure, where a base plate (19) contains the sensor devices (1), the wiring and an electronic circuit device. The top part (20) is laminated on the base plate, whereafter the base plate and the top part together form the implantable device.

In the top part (20) two spaces (21) and (22) are made, simply by removing some material from the top part (20). The two spaces form two areas so that the device is able to detect light from two areas. The space (21) is created on the surface of the top part which faces away from the base plate so that compounds and tissue have access to the space when the device is implanted. The space (22), however, is formed on the surface of the top part which faces towards the base plate so that compounds and tissue have no access to the space (22) when the device is implanted, as the space (22) is closed. This is indicated in FIG. 10, showing a section through X-X of the top part of FIG. 9. The detecting area underneath space (21) and space (22) is formed on the same surface but as the space (22) is closed, the interaction of light with compounds and tissue occurs over a larger distance at space (21) than at space (22). The closed space (22) forms a spacer. A spacer can also be formed of a solid material transparent to the incident light. A “spacer” is to be understood as a volume in which no interaction of light with compounds and tissue occurs.

Instead of operating with transmitted light received by photo detectors, the photo detectors can be replaced by reflectors to reflect light back to external photo detectors for analysis, or more specifically for the present invention, to reflective surfaces with micro structures as it shall be described in the following.

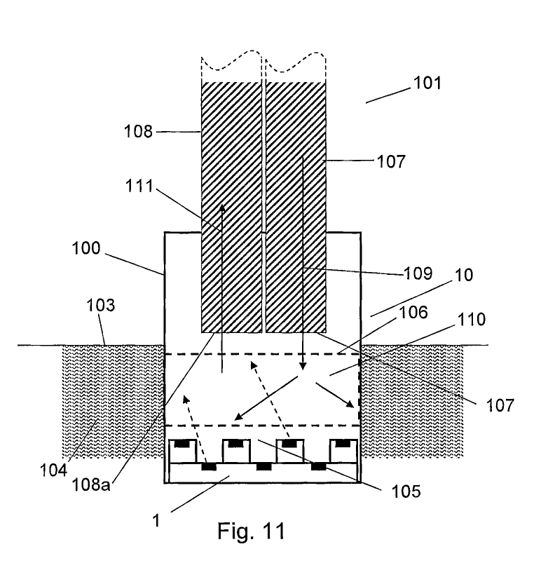

FIG. 11 shows one basic embodiment of a system of the present invention. The system (101) comprises a detection device in the shape of a probe head (102) arranged partly subcutaneously, i.e. below a skin surface (103). Thereby the probe head (102) is arranged in direct contact with tissue (104) comprising interstitial fluid present in this region. It should be noted that the probe head (102) could, alternatively, be arranged in a blood vessel or in contact with a suitable body fluid being sampled separately, e.g. via a catheter.

Inside the probe head (102) an analysis volume (105) is defined. The analysis volume (105) is partly delimited towards the interstitial fluid by a semi-permeable membrane (106) positioned in any manner as a part of the walls (100) separating the analysis volume (105) from the surrounding tissue (104). The semi-permeable membrane (106) could in this embodiment be anything like a ‘window’ in the walls (100), or a band encircling the probe head (102), or the whole of the walls (100) enclosing the analysis volume (105) optionally could be a semi-permeable membrane (106). The semi-permeable membrane (106) allows substances of interest, such as glucose, to pass, while other constituents of the body fluid are not allowed to pass the semi-permeable membrane (106). Accordingly, molecules of a particular substance of interest are present in the analysis volume (105).

The system (101) further comprises a first light guide (107) and a second light guide (108), both having an end (107a), (108a) arranged in the analysis volume (105). The first light guide (107) is arranged to guide primary light (109) to the analysis volume (105). In the analysis volume (105) the primary light (109) is scattered from molecules of the substance of interest, e.g. due to Raman scattering, thereby producing secondary light in the form of scattered light (110). The second light guide (108) is arranged to guide secondary light (111) away from the analysis volume and towards a detection unit and/or an analysis unit, in which the secondary, scattered light (111) is detected and/or analysed and used as a basis for determining the concentration of the substance of interest in the body fluid being analysed.

The analysis volume (105) further comprises a sensor device (1) as in any of the embodiments previously described, where at least one of the first or second substantially flat areas, or at least one member of the first and second group of areas, comprises an optically responsive element (8), in the following context meaning any device or element actively or passively reacting to light like a photo detector or just a surface reflecting part or all of the light hitting the surface.

Especially if the optically responsive element (8) comprises a reflective part or surface, then, when primary light (109) enters the analysis volume (105) via the first light guide (107), the part which is not scattered immediately, is reflected from the reflective surface. Thereby the primary light (109) travels the distance of the analysis volume (105) once again, and the probability of a given photon being scattered by a molecule of the substance of interest is thereby doubled. Thereby an enhanced signal is obtained.

In a more preferred embodiment, at least one of the optically responsive elements (8) is a reflective surface being a structured metal surface, e.g. a microstructured metal surface. More preferably, all of the optically responsive elements (8), or at least just all the optically responsive elements (8) at one of the first or second group of areas. The structured metal surface is preferably of a kind which is suitable for adsorption of molecules of the substance of interest. In this case an enhanced Raman signal can be obtained by means of Surface Enhanced Raman Spectroscopy (SERS). Thereby it is possible to detect the presence of substances of interest even at very low concentrations.

The structured metal surface may advantageously be made from a precious metal, such as gold, silver, copper or platinum.

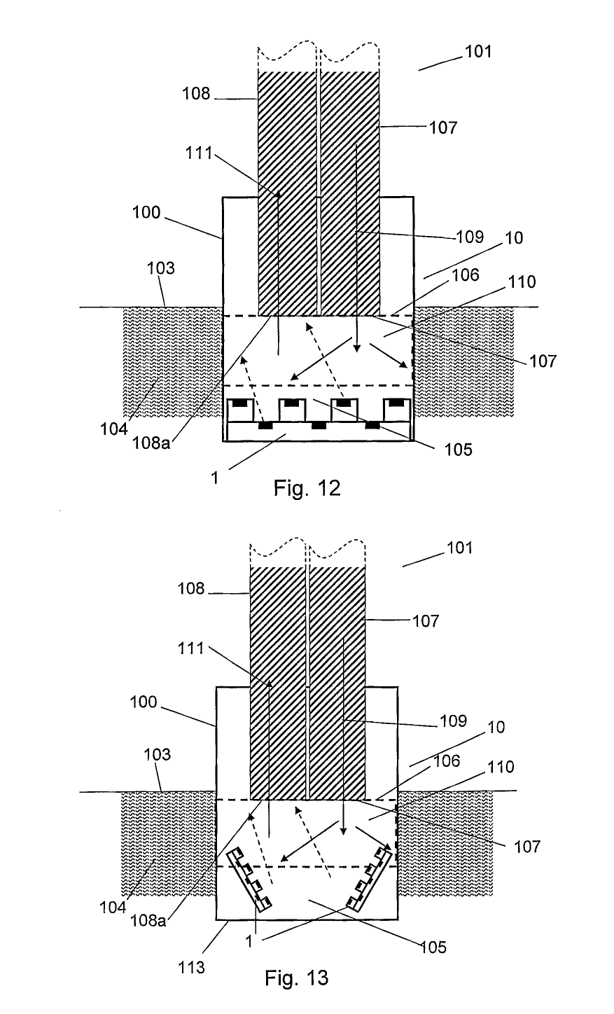

FIG. 12 is a schematic view of a system (101) according to an embodiment very similar to the system (101) of FIG. 11. However, in the embodiment of FIG. 12 the ends (107a), (108a) of the light guides (107), (108) are arranged at a position corresponding to the position of the semi-permeable membrane (106), and below the skin surface (103).

FIG. 13 is a schematic view of a system (101) according to a further embodiment of the invention. The system (101) of FIG. 13 is very similar to the system of FIG. 11. However, in this system optically responsive elements (8) are arranged in the analysis volume (105) at an angle to the end part (113) of the probe head (102). The optically responsive elements (8) may be in the form of a plurality of elements, or it may be a single surface having a substantially cylindrical shape.

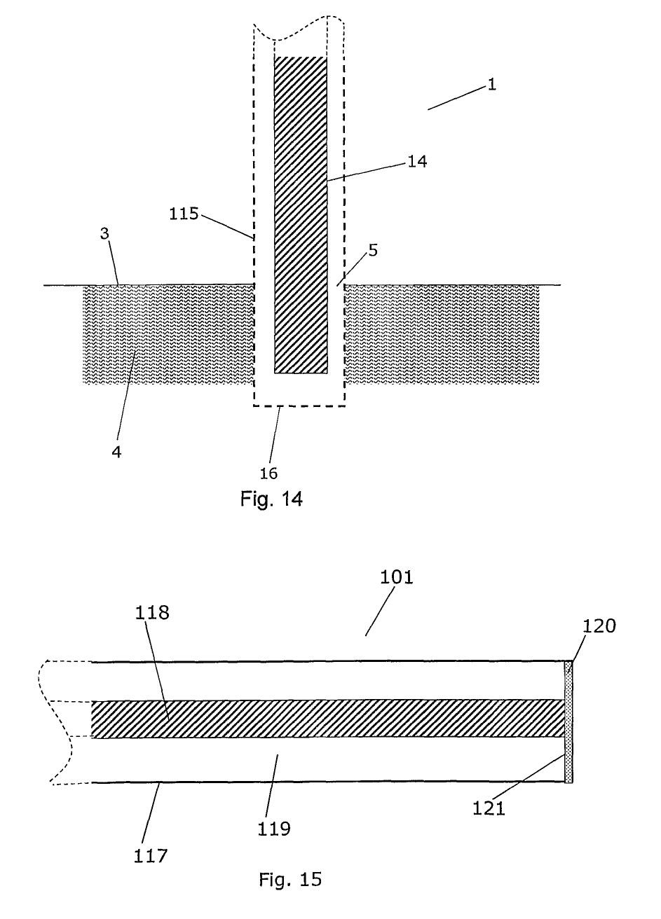

FIG. 14 is a schematic view of a system (101) according to another embodiment of the invention. The system (101) of FIG. 14 comprises a light guide in the form of an optical core (114) arranged inside a hollow fibre (115) made from a semi-permeable material. An end part (116) of the hollow fibre (115) is arranged beneath the skin surface (103), and the semi-permeable membrane material of the hollow fibre (115) is thereby in direct contact with the tissue (104). Accordingly, molecules of a substance of interest, e.g. glucose, are thereby allowed to enter a space defined between the optical core (114) and the hollow fibre (115). This space thereby constitutes an analysis volume (105).

Furthermore, the analysis volume (105), delimited by the semi-permeable hollow fibre (115), forms a region having a lower index of refraction than the optical core (114). Thereby the analysis volume (105) may act as a cladding layer. In this case the optical core (114) and the analysis volume (105) in combination form a concentric optical waveguide which can be used to guide primary light towards the end part (116) as well as to guide secondary light away from the end part (116). Primary light arriving at the analysis volume (105) is scattered, e.g. Raman scattered, on molecules of the substance of interest. The scattered light is guided away from the analysis volume and towards a detection and/or analysis apparatus via the waveguide formed by the optical core (114) and the analysis volume (105). This is similar to the embodiments described above.

The system (101) shown in FIG. 14 has the advantage that the region where primary light can be scattered on molecules of the substance of interest is relatively large. Thereby a larger signal can be obtained, and it is possible to detect smaller concentrations of the substance of interest.

The analysis volume (105), in particular the part arranged near the end part (116), may be provided with sensor device (1) according to any of the previously described embodiments.

FIG. 15 is a schematic view of a system (101) according to even a further embodiment of the invention. The system (101) of FIG. 15 comprises an optical fibre (117) comprising a core (118) and a cladding layer (119). A sensor device (1) according to any of the preceding embodiments is attached at an end part (121) of the optical fibre (117).

The system of FIG. 15 is preferably operated in the following manner. At least the end part (121) of the optical fibre (117) along with the metal microstructure (120) is positioned in contact with a body fluid to be analysed. The system may be arranged invasively, e.g. subcutaneously as in the previously described embodiments, or it may be arranged in contact with a previously obtained sample.

Especially when the sensor device (1) comprises microstructures as previously described, these metal microstructures are then arranged in direct contact with the body fluid, and thereby with molecules of the substance of interest. Thereby molecules of the substance of interest are adsorbed on the surface of the metal microstructure.

Primary light is guided by means of the optical fibre (117) to the metal microstructure. Here it is scattered, preferably Raman scattered, on the molecules of the substance of interest which have been adsorbed on the surface of the metal microstructure. The scattered light is guided away from the metal microstructure (120) and towards a detection and/or analysis unit by means of the optical fibre (117). Here the concentration of the substance of interest is determined. Since the primary light was scattered on molecules of the substance of interest which were adsorbed on the metal microstructure, this determination can be made using Surface Enhanced Raman Spectroscopy (SERS). As described above, a stronger signal can thereby be obtained, allowing smaller concentrations of the substance of interest to be detected.

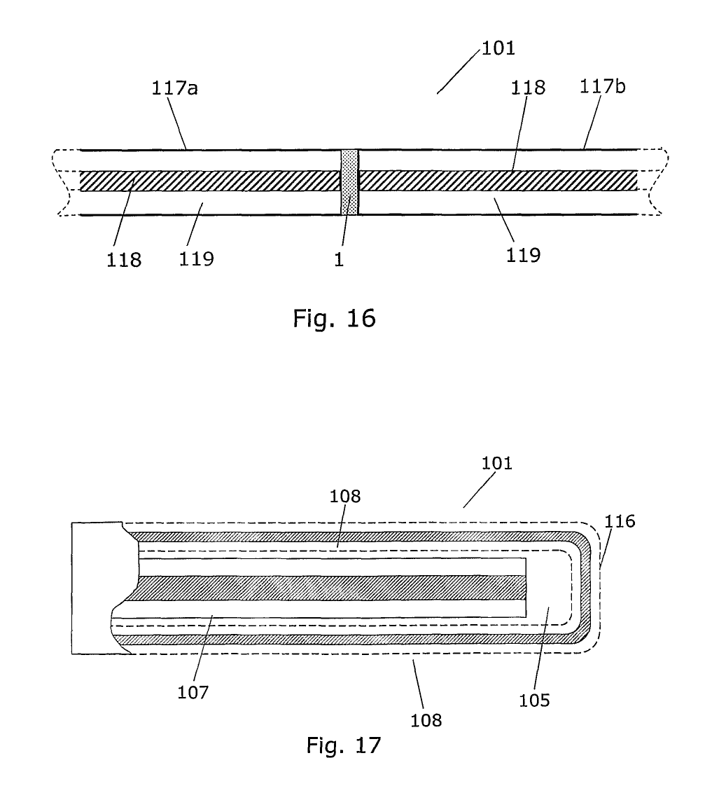

FIG. 16 is a schematic view of a system (101) according to an embodiment of the invention very similar to the system (101) shown in FIG. 15, only a part of the optical fibre (112) has been removed and a sensor device (1), preferably comprising a metal microstructure, has been arranged at the position where material has been removed. It should be noted that all of the fibre material at this position could be removed, in which case only the metal microstructure keeps the two parts (117a), (117b) of the optical fibre (117) together. Alternatively, only part of the fibre material may be removed, e.g. one or more segments of the optical fibre (117), or only the cladding material (119), leaving the core (118).

The system (101) of FIG. 16 is preferably operated in the following manner. The part of the optical fibre (117) where the metal microstructure is positioned is arranged in contact with a body fluid to be analysed. The optical fibre (117) may be arranged invasively, e.g. subcutaneously as described above, or it may be arranged in contact with a previously obtained sample. In the case that the optical fibre (117) is arranged invasively it could be envisaged that the skin surface is penetrated in two positions, a first part (117a) of the optical fibre (117) protruding through one of the penetrations and a second part (117b) of the optical fibre (117) protruding through the other penetration, thereby positioning the metal microstructure invasively and in contact with the body fluid and thereby in contact with the substance of interest.

Molecules of the substance of interest will then be adsorbed on the metal microstructure as described above, and the metal microstructure defines an analysis volume.

Primary light is guided towards the metal microstructure via the first part (117a) of the optical fibre (117). Some of the primary light is scattered on the adsorbed molecules of the substance of interest, and the scattered light is guided away from the metal microstructure via the second part (117b) of the optical fibre (117). The scattered light is analysed using Surface Enhanced Raman Spectroscopy (SERS) as described above.

FIG. 17 is a schematic view of a system (101) according to an embodiment very similar to the system (101) shown in FIG. 11 or 12. The system (101) of FIG. 17 comprises a first light guide (107) arranged to guide primary light towards an analysis volume (105) and a second light guide (108) arranged to guide secondary light away from the analysis volume (105). The second light guide (108) is made from a semi-permeable material, i.e. molecules of the substance of interest are allowed to pass through the second light guide (108) and into the analysis volume (105).

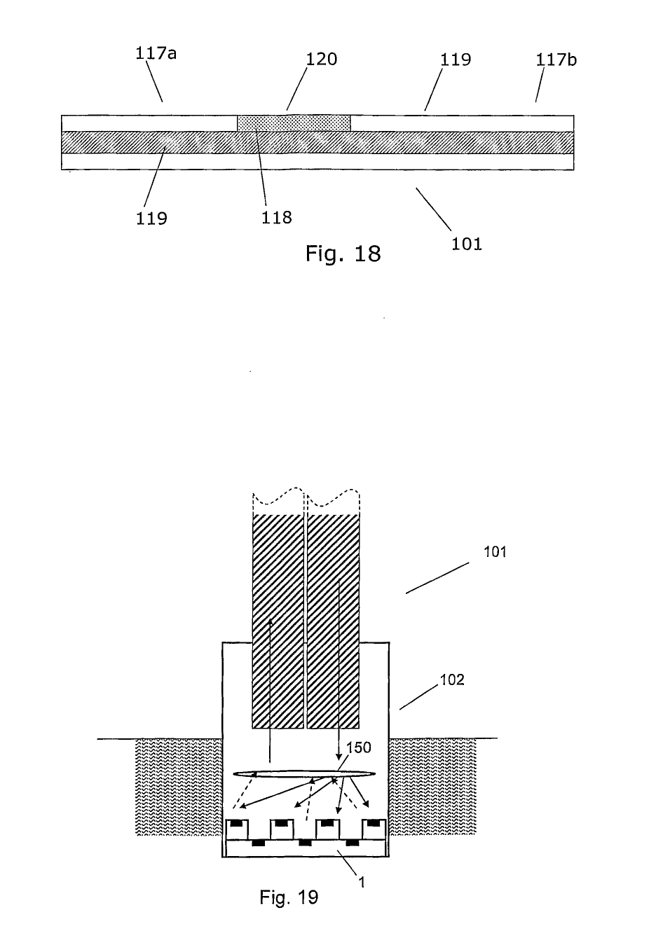

FIG. 18 is a schematic view of a system (101) according to a further embodiment of the invention. In the system (101) of FIG. 18 only part of the cladding layer (119) has been removed and a metal microstructure has been arranged at this position. The metal microstructure forms an analysis volume.

FIG. 19 shows an embodiment where the probe head (102) comprises a lens system (150) to scatter and/or focus light to and/or scattered from the sensor device (1).

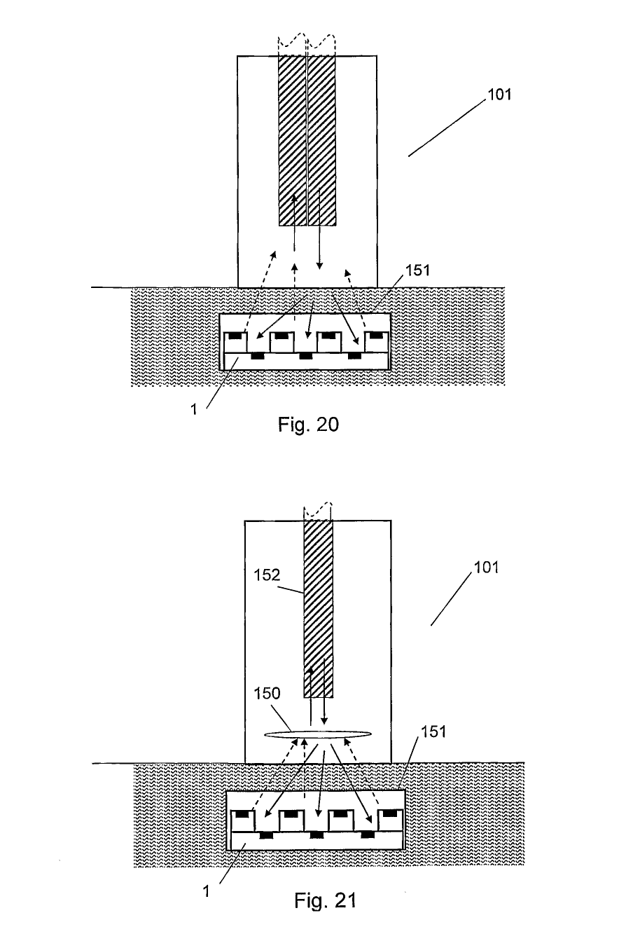

FIG. 20 shows an embodiment, where the sensor device (1) is not connected to the light guides, but is implanted as part of an implanted device (151) as also seen in FIG. 1.

FIG. 21 shows an embodiment, where the sensor device (1) is not connected to the light guides, but is implanted as part of an implanted device (151) as also seen in FIG. 1. The primary and secondary light then penetrates the skin. The light guides in this and any of the above embodiments could be a single light guide (152), such as an optical fibre, guiding both primary and secondary light. The guide system optionally could comprise a lens (150).

While the present invention has been illustrated and described with respect to a particular embodiment thereof, it should be appreciated by those of ordinary skill in the art that various modifications to this invention may be made without departing from the spirit and scope of the present.

What is claimed is:

1.-21. (canceled)

22. A system for determining a concentration of a substance of interest in a body fluid, the system comprising:
a sensor device adapted to be positioned in direct contact with a body fluid to be analysed, said sensor device defining an analysis volume, at least partly delimited towards the body fluid by a semi-permeable membrane allowing substances of interest from the body fluid to enter the analysis volume,
a light source adapted to transfer primary light to the sensor device,
wherein the sensor device comprises at least one substantially flat area constituting a first area and at least one substantially flat area constituting a second area, at least one area comprising an optically responsive element,
wherein groups of areas are provided, each group of areas forming a common level and the common levels having a predetermined spacing from each other to provide the said difference in distance over which light interacts with compounds and tissue and wherein the first group of areas is formed at a base level of the device and the second group of areas is formed by projections from the said base level to a top level.

23. The system according to claim 22, wherein at least one of the first or second substantially flat areas, or at least one member of the first and second group of areas, comprises an optically responsive element comprising any number and combination of a SERS, a photo detector and a reflective surface.

24. The system according to claim 22, further comprising first light guiding means arranged to guide primary light from the light source to the analysis volume.

25. The system according to claim 24, further comprising second light guiding means arranged to guide secondary light away from the analysis volume.

26. The system according to claim 22, wherein the sensor device forms part of a probe head adapted to be positioned invasively, a semi-permeable membrane being arranged in a wall part of the probe head.

27. The system according to claim 26, wherein at least one of the first and second light guiding means is positioned with an end face in contact with the internal of the probe head.

28. A device according to claim 22, wherein at least a part of at least one of said areas is covered by a permeable membrane forming an analysis volume.

29. A device according to claim 22, wherein the sensor device is positioned/implanted under the skin of an animal.

30. The device according to claim 29 wherein the animal is a human, the sensor device being positioned/implanted under the skin of a human being.

31. The device according to claim 29, wherein the light source and possibly first and/or second light guiding means are external to the animal/human body, all transmitted and/or reflected light penetrating the skin of the animal/human body.

32. The system according to claim 22, further comprising at least one laser for emitting primary light, said laser(s) being connected to the first light guiding means.

33. The system according to claim 22, further comprising detection means adapted to detect Raman scattered light, said detection means being connected to the second light guiding means.

34. The system according to claim 22, wherein the substance of interest is glucose.

35. The system according to claim 22, wherein the body fluid is blood.

36. The system according to claim 22, wherein the first and the second light guiding means comprise(s) an optical fibre.
